(19) United States
(12) Patent Application Publication
(10) Pub. No.: US 2009/0247890 A1
(43) Pub. Date: Oct. 1, 2009

(54) SOLID STATE MYOCARDIAL INFARCTION DETECTOR

(75) Inventors: Gilbert Hausmann, Felton, CA (US); Michael P. O'Neil, Danville, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/365,295

(22) Filed: Feb. 4, 2009

Related U.S. Application Data
(60) Provisional application No. 61/039,696, filed on Mar. 26, 2008.

(51) Int. Cl. A61B 5/097

(52) U.S. Cl. 600/532

(57) ABSTRACT

The disclosure, according to one embodiment, relates to a breath analyzer device for detecting concentration of a marker, such as pentane, in air exhaled by a patient. The device may include a detector having a solid state sensor able to detect pentane in a concentration of between 1 nmol/l to 5 nmol/l in air exhaled by a patient. The solid state sensor may include a carbon nanotube. The breath analyzer may include a body having a breath detection channel in which the detector is located, a display, and a power source. The disclosure also relates to a method of detecting myocardial infarction in a patient by detecting pentane in exhaled air from the patient.

### CROSS-REFERENCE TO RELATED APPLICATION

This claims the benefit of U.S. Provisional Patent Application No. 61/039,696, filed Mar. 26, 2008, which is hereby incorporated by reference herein in its entirety.

### TECHNICAL FIELD

The present disclosure, according to one embodiment, relates to a device for detecting myocardial infarction. In particular embodiments, it relates to a device for detecting pentane or other markers in exhaled air. The device, according to another embodiment, may be a sensor including a solid state detector. In a particular embodiment, the solid state detector may include carbon nanotubes.

### BACKGROUND

Rapid and accurate detection of acute myocardial infarction can be critical to proper and prompt treatment and patient survival. Some current detection methods, such as various heart monitors, are effective, but are only easily used in controlled settings, such as a hospital bed. Emergency detection, particularly as used in emergency vehicles and otherwise outside of a hospital is sometimes inadequate. Additionally, emergency detection methods may sometimes produce an incorrect diagnosis, resulting in inappropriate care. Many patients suffer acute myocardial infarction outside of a hospital and would benefit from improved or more widely available detection devices. Hospitalized patients might also benefit from these devices.

Patients with acute myocardial infraction often exhale a distinctive and elevated level of certain substances. For example, levels of pentane are increased in the exhaled air of these patients.

### SUMMARY

Therefore, there is a need for a device able to detect pentane and or other markers of myocardial infarction in the exhaled air of patients suffering or suspected of suffering acute myocardial infarction.

According to one embodiment, the disclosure relates to a device for detecting pentane concentration in exhaled air including a detector having a solid state sensor able to detect pentane in a concentration of between 1 nmol/l to 5 nmol/l in air exhaled by a patient. In a more particular embodiment, the solid state sensor may include a carbon nanotube. In another particular embodiment, the device may also include a body having a breath detection channel in which the detector is located, a display, and a power source,

According to another embodiment, the disclosure relates to a device for detecting pentane concentration in exhaled air using a means for detecting pentane.

According to a third embodiment, the disclosure relates to a method of detecting myocardial infarction in a patient by directing exhaled air from the patient into a breath analyzer of any type described above. The method may also include detecting pentane in a concentration of between 1 nmol/l to 5 nmol/l in the exhaled air and providing a visual or audible display of pentane concentration in the exhaled air. Pentane concentration may be indicative of the presence or absence of acute myocardial infarction.

### BRIEF DESCRIPTION OF THE DRAWINGS

A more complete understanding of the present disclosure may be acquired by referring to the following description taken in conjunction with the accompanying drawings.

FIG. 1 illustrates a breath analyzer apparatus for diagnosing acute myocardial infarction according to an embodiment of the present disclosure. FIG. 1a is a front view of the breath analyzer. FIG. 1b is a side view.

### DETAILED DESCRIPTION

Referring now to the drawings, the details of specific example embodiments are schematically illustrated. Like elements in the drawings are represented by like numbers.

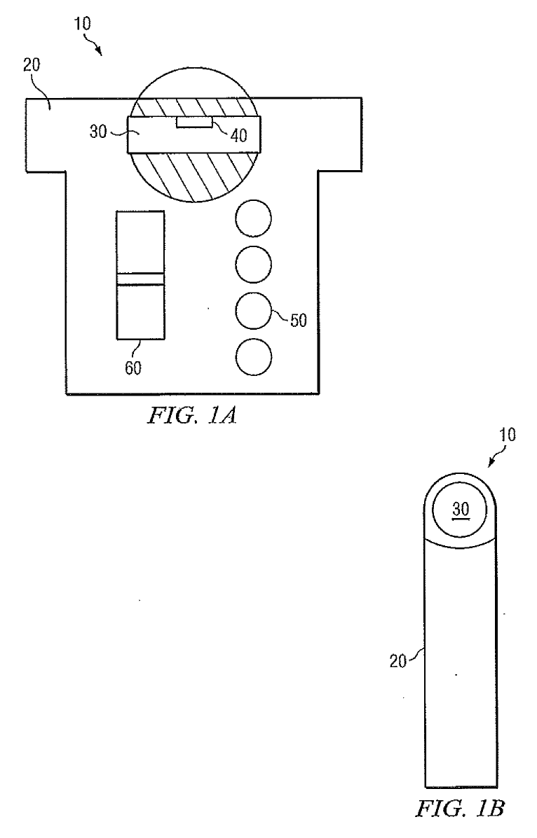

FIG. 1 illustrates a breath analyzer device 10 for detecting acute myocardial infarction. Breath analyzer device 10 includes a body 20 containing breath detection channel 30. Solid state detector 40 may be located in breath detection channel 30. Solid state detector 40 may be connected to a processor/circuitry (not shown) which processes a signal from detector 40 to provide an output at a display, such as indicator 50. Breath analyzer device 10 may also include on/off switch 60. Breath analyzer device 10 may also include a power source (not shown) such as a battery or electrical connection. The power source may be selected, for example, based on whether or not the breath analyzer 10 is mobile or stationary.

The levels of pentane found in exhaled air of patients suffering from acute myocardial infarction are described at least in Weitz, Z W et al. “High breath pentane concentration during acute myocardial infarction,” Lancet, 337, pp. 933-5 (1991). In particular, pentane levels may be between 4-5 nmol/l in exhaled air when a patient is suffering acute myocardial infarction. In contrast, in healthy patients, or those not suffering myocardial infarction, the pentane levels in respired air are typically less than 2 nmol/l. Accordingly, breath analyzer device 10 may be able to distinguish pentane levels in respired air in a range of between 1 nmol/l and 5 nmol/l. Breath analyzer device may also be able to accurately distinguish differences in pentane concentration, particularly in this range, of 2 nmol/l, 1 nmol/l or 0.5 nmol/l.

Breath analyzer 10 may also be able to distinguish other exhaled markers of myocardial infarction. For example, it is well known that serum troponin levels increase during acute myocardial infarction. Troponin, breakdown products of troponin, or other troponin-linked markers may be exhaled during acute myocardial infarction and detected. Breath analyzer 10 may be designed to be able to detect these markers in a range spanning normal marker levels and levels present during acute myocardial infarction. It may also be able to accurately distinguish differences in marker concentration sufficient to distinguish a patient suffering acute myocardial infarction from one not.

Detector 40, in one embodiment, may include a chemo-electrical sensor element that contains a solid body of a material. The solid body of material may change electrical properties in the presence of the marker in a predictable and repeatable manner. For example, the solid body may contain a material that converts the presence and concentration of the exhaled marker, such as pentane, in a given gas volume to free electrons. For example, detector 40 may include carbon nanotubes. In one embodiment using carbon nanotubes, a marker-sensitive layer of polymer may act as an electron donor, increasing the conductivity of an underlying layer of carbon nanotubes, which may be randomly oriented. The nanotubes may then form the channel of a field effect transistor structure. The channel current may then generate a predictable signal representing concentration of the marker. This electrical signal may then be processed by an analog to digital converter to produce a digital signal. The digital signal may then be further processed, for example by software stored in nonvolatile memory and able to translate the digital signal into information about marker concentration.

Nanotubes used to detect the marker may be similar to the types produced by Nanomix, Inc. (Emeryville, Calif.), particularly as described in Star et al., Nanoelectric Carbon Dioxide Sensors, Advanced Materials, 16:22, pp. 2049-2052 Nov. 18, 2004), US2004/0132070, US2004/0043527, US2003/0175161, and US2003/0134433, or by Nanohorizons, Inc. (State College, Pa.) all of which are incorporated by reference herein.

Detector 40 may include a single solid state sensor or an array of solid state sensors. It may also detect multiple markers. Detector 40 may be replaceable.

Breath analyzer 10 may be sized to be mobile or stationary, depending on the intended use. Mobile breath analyzers may be particularly useful in diagnosing acute myocardial infarction in emergency situations in the field. For example, breath analyzer 10 may be a handheld device.

Body 20 with breath detection channel 30 may be designed to allow it to house detector 40, a display, and signal amplification, conversion, or processing components. Body 20 and breath detection channel 30 may be designed to place detector 40 in the gas path of air exhaled by a patient. Body 20 may be shaped to allow for a sufficient lip seal to obtain a sufficiently high sample availability or flow rate in breath detection channel 30. Body 20 may be designed for attachment to one or more tubes or other devices to direct exhaled air from the patient to breath detection channel 30. Body 20 may also, alternatively, have a housing to be inserted in a patient's nose or mouth to direct exhaled air into breath detection channel 30. Any tubes or housing may be designed to be disposable and may be provided in sterile packaging. Breath detection channel 30 may be designed to allow inhaled air to flow though it as needed.

Detector 40 may produce an analog signal, for example via the solid state component, which may be amplified. The analog signal may be converted to a visually or acoustically recognizable signal indicating marker concentration in exhaled air. In one embodiment, it may be converted to a digital signal. The digital signal may then be processed to determine concentration of the marker. This signal processing may involve software run on a CPU that uses a RAM/ROM component for information storage. Analog or digital components may also be capable of calibrating breath analyzer 10 or performing various self-checking procedures. Analog or digital components may also indicate when detector 40, if replaceable, should be replaced. Replacement may be indicated based on the amount of time detector 40 has been in breath analyzer 10, the number of times detector 40 has been used, or other indicators of detector 40 function.

The display may be an indicator, such as indicator 50, which provides a simple visual signal, such as red or green or shades or yellow or orange, of whether or not marker levels are safe, indicative of acute myocardial infarction, or close to an unsafe level. Acoustic indicators may be used in place or in addition to the display. Alternatively, the display may provide a simple visual signal or it may show the actual measured concentration of the marker. The display may also provide suggestions for treatment options.

Although an example breath analyzer device is shown in FIG. 1, one ordinarily skilled in the art will readily understand that solid state detector 40 may be incorporated in a variety of breath analyzer devices.

Acute myocardial infarction may be detected in a patient using a breath analyzer such as breath analyzer device 10. Body 20 may be connected to exhaled air from the patient, for example via a tube or placement in the patient's mouth. The presence of a marker of myocardial infarction, such as pentane creates a detectable electrical signal or signature of electrical signals in detector 40. This signal or these signals may then trigger a visual or audible notification of marker concentration. In some embodiments, notification may occur quickly after exhaled air enters the breath analyzer. For example, notification may be near instantaneous, within 1 second, within 5 seconds, or within 10 seconds.

The patient may be any individual, human or animal, who is experiencing or is suspected of experiencing myocardial infarction.

While embodiments of this disclosure have been depicted, described, and are defined by reference to specific embodiments of the disclosure, such references do not imply a limitation of the disclosure, and no such limitation is to be inferred. The subject matter disclosed is capable of considerable modification, alteration, and equivalents in form and function, as will occur to those ordinarily skilled in the pertinent art and having the benefit of this disclosure. The depicted and described embodiments of this disclosure are examples only, and are not exhaustive of the scope of the disclosure. For example, one skilled in the art may readily imagine a variety of permutations in which the breath analyzer may be configured. A variety of ways of communicating information may also be imagined.

What is claimed is:

1. A device for detecting pentane concentration in exhaled air comprising a detector comprising a solid state sensor able to detect pentane in a concentration of between 1 nmol/l to 5 nmol/l in air exhaled by a patient.

2. The device according to claim 1 wherein the solid state sensor comprises a carbon nanotube.

3. The device according to claim 2, comprising:
a pentane-sensitive layer of polymer; and
an underlying layer of carbon nanotubes.

4. The device according to claim 1 further comprising:
a body comprising a breath detection channel in which the detector is located;
a display; and
a power source.

5. The device according to claim 1 further comprising a processor or circuitry operable to process a signal from the detector and provide information to the display.

6. The device according to claim 1, wherein the display is a visual indicator.

7. The device according to claim 1, wherein the display is an audible indicator.

8. The device according to claim 1, wherein the device is mobile and the power source is a battery.

9. The device according to claim 1, wherein the device is stationary and the power source is an electrical connection.

10. A device for detecting pentane concentration in exhaled air comprising a means for detecting pentane.

11. The device according to claim 10 further comprising:
a housing means for housing the means for detecting pentane;
a means for displaying or indicating pentane concentration; and
a means for supplying power.

12. The device according to claim 10 further comprising a means to transmit a signal from the means for detecting pentane to the means for displaying or indicating the pentane concentration.

13. The device according to claim 10 further comprising means to process a signal from the means for detecting pentane.

14. A method of detecting myocardial infarction in a patient comprising:
directing exhaled air from the patient into a breath analyzer comprising a detector comprising a solid state sensor able to detect pentane in a concentration of between 1 nmol/l to 5 nmol/l in the exhaled air;
detecting pentane in a concentration of between 1 nmol/l to 5 nmol/l in the exhaled air;
providing a visual or audible display of pentane concentration in the exhaled air, wherein pentane concentration is indicative of the presence or absence of acute myocardial infarction.

15. The method according to claim 14 further wherein the visual display shows the measured concentration of pentane.

16. The method according to claim 14, further comprising producing an analog signal that corresponds to pentane concentration in the exhaled air.

17. The method according to claim 16, further comprising providing the visual or audible display based directly on the analog signal.

18. The method according to claim 16, further comprising converting the analog signal to a digital signal.

19. The method according to claim 18, further comprising processing the digital signal to produce information about pentane concentration.

20. The method according to claim 18, further comprising providing the visual or audible display based on the digital signal.
